(19) United States
(12) Patent Application Publication
(10) Pub. No.: US 2022/0096452 A1
(43) Pub. Date: Mar. 31, 2022

(54) LOCAL ANESTHETIC FOR THE TREATMENT OF NEUROLOGICAL SYMPTOMS RESULTING FROM BRAIN DYSFUNCTIONS

(71) Applicants: ICM (INSTITUT DU CERVEAU ET DE LA MOELLE ÉPINIÈRE), Paris (FR); ASSISTANCE PUBLIQUE HOPITAUX DE PARIS, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MÉDICALE), Paris Cedex 13 (FR); SORBONNE UNIVERSITE, Paris (FR)

(72) Inventors: Laurent COHEN, Paris (FR); Randa EL HACHEM, Narbonne (FR); Piirika CREPIN, Narbonne (FR); Guillaume GUERINEAU DE LAMERIE, Saint-Sulpice-la-Forêt (FR); Gérard SYLVESTRE, Lézignan-Corbières (FR)

(73) Assignees: ICM (INSTITUT DU CERVEAU ET DE LA MOELLE ÉPINIÈRE), Paris (FR); ASSISTANCE PUBLIQUE HOPITAUX DE PARIS, Paris (FR); CENTRE NATIONAL DE LA RECHERCHE SCIENTIFIQUE, Paris (FR); INSERM (INSTITUT NATIONAL DE LA SANTÉ ET DE LA RECHERCHE MÉDICALE), Paris Cedex 13 (FR); SORBONNE UNIVERSITE, Paris (FR)

(21) Appl. No.: 17/422,554

(22) PCT Filed: Jan. 22, 2020
(86) PCT No.: PCT/EP2020/051491

§ 371 Date: Jul. 13, 2021

(30) Foreign Application Priority Data
Jan. 22, 2019 (EP) 19305079.6

(51) Int. Cl. A61K 31/445; A61K 31/167; A61K 31/245; A61K 31/606; A61K 31/4458; A61K 31/46; A61K 31/235; A61K 31/4525; A61P 25/28

(52) U.S. Cl. CPC A61K 31/445; A61K 31/167; A61K 31/245; A61K 31/606; A61P 25/28; A61K 31/46; A61K 31/235; A61K 31/4525; A61K 31/4458

(57) ABSTRACT

A local anesthetic chosen from the group of amino amide or amino ester, and a composition or pharmaceutical composition including the local anesthetic. The local anesthetic is for use in the treatment of neurological symptoms resulting from brain dysfunctions such as motor, cognitive, sensory, visual deficits, and/or apathetic, catatonic, vegetative and/or minimally conscious states.

### FIELD OF INVENTION

The present invention relates to the use of local anesthetics chosen from the group consisting of amino amides and amino esters for the treatment of neurological symptoms resulting from brain dysfunctions.

### BACKGROUND OF INVENTION

The management of patients with neurological symptoms resulting from brain or dysfunctions, such as visual scotoma, motor deficits, sensory deficits, language or other cognitive impairments is largely restricted to symptomatic care.

The means to effectively treat these symptoms are extremely limited. To date, several avenues have been explored: cognitive stimulation, physical and speech therapy, transcranial magnetic or electrical stimulation, stimulation via electrodes implanted in deep brain structures, pharmacological intervention on various neurotransmitter systems.

To date, all these approaches remain disappointing, and there is still a major need for effective therapeutic means.

The present invention results from the serendipitous discovery that the administration of a local anesthetic to a subject in need thereof allows for the transient recovery of neurological symptoms resulting from stroke.

### SUMMARY

This invention thus relates to a local anesthetic chosen in the group consisting of amino amides, amino esters and any combination thereof for use in the treatment of neurological symptoms resulting from brain dysfunctions.

This invention relates to the administration of a local anesthetic chosen in the group consisting of amino amides, amino esters and any combination thereof for inducing the transient and/or partial recovery of neurological symptoms resulting from brain dysfunctions.

According to a first aspect, the local anesthetic of the invention is to be used where the symptoms of brain dysfunctions result from stroke, tumor, head trauma, inflammatory diseases of the central nervous system, degenerative diseases, depression or psychosis.

According to another aspect, the local anesthetic of the invention is to be used where the brain dysfunctions results in motor, cognitive, sensory, visual deficits and/or in apathetic, catatonic, vegetative, minimally conscious states.

In one embodiment, the local anesthetic is an amino-amide chosen among bupivacaine, levo-bupivacaine, carticaine, lidocaine, prilocaine, etidocaine, levo-etidocaine, dextro-etidocaine, mepivacaine, levo-mepivacaine, ropivacaine, levo-ropivacaine, trimecaine, pyrrocaine, dibucaine, lidocaine salicylate monohydrate, octacaine, oxethazaine, pipecoloxylidides, sameridine, articaine, aptocaine, QX-314, 2-alkyl-2-alkylamino-2′, 6′acetoxylidide compounds such as those described in U.S. Pat. No. 3,862,321, tertiaryalkylamino-lower acyl-xylidide compounds such as those described in U.S. Pat. No. 3,925,469; 2(ω-alkylaminoalkyl)-3-(4-substituted-benzylidene) phthalimidine compounds or 2-(ω-dialkylaminoalkyl)-3-(4-substituted-benzylidene) phthalimidine compounds such as those described in U.S. Pat. No. 4,551,453; L-N-n-propylpipecolic acid-2,6-xylidide; N-substituted 4-piperidinecarboxamide compounds such as those described in U.S. Pat. No. 5,756,520; N-substituted 4-phenyl-4-piperidinecarboxamide compounds, such as those described in U.S. Pat. No. 5,360,805; compounds of formula (II):

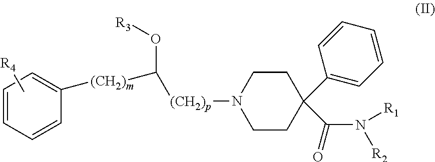

wherein R₁₋₄, m, and P are defined as in International Patent Application Publication No. WO 95/21821, and/or analog and/or functional derivative thereof and/or any combination thereof.

2-alkyl-2-alkylamino-2′, 6′acetoxylidide compounds such as those described in U.S. Pat. No. 3,862,321 include 2-diethylamino-2′,6′-n-butyroxylidide (which can be alternatively named 2-ethyl-2-diethylamino-2′,6′-acetoxylidide), 2-(N-ethyl-n-propylamino)-2′,6′-n-butyroxylidide (which can be alternatively named 2-ethyl-2-(N-ethyl-n-propylamino)-2′,6′-acetoxylidide), 2-diethylamino-2′,6′-n-valeroxylidide (which can be alternatively named 2-n-propyl-2-diethylamino-2′,6′-acetoxylidide), 2-pyrrolidino-2′,6′-n-butyroxylidide (which can be alternatively named 2-ethyl-2-pyrrolidino-2′,6′-acetoxylidide), 2-(N-ethyl-n-butylamino)-2′,6′-n-butyroxylidide (which can be alternatively named 2-ethyl-2-(N-ethyl-n-butylamino)-2′,6′-acetoxylidide), 2-dimethylamino-2′,6′-caproylxylidide (which can be alternatively named 2-n-butyl-2-dimethylamino-2′,6′-acetoxylidide) and 2-pyrrolidino-2′,6′-n-valeroxylidide (which can be alternatively named 2-n-propyl-2-pyrrolidino-2′,6′-acetoxylidide).

Tertiaryalkylamino-lower acyl-xylidide compounds such as those described in U.S. Pat. No. 3,925,469 include 2-(tert.-butylamino)-2′,6′-acetoxylidide, 2-(tert.-butylamino)-2′,6′-propionoxylidide, 2-(N-ethyl-tert.-amylamino)-2′,6′-acetoxylidide, 2-(N-n-propyl-tert.-amylamino)-2′,6′-acetoxylidide, 2-(N-n-butyl-tert. butylamino)-2′,6′-acetoxylidide and 2-(N-tert.-amylamino)-2′,6′-acetoxylidide.

2(ω-alkylaminoalkyl)-3-(4-substituted-benzylidene) phthalimidine compounds or 2-ω-dialkylaminoalkyl)-3-(4-substituted-benzylidene) phthalimidine compounds such as those described in U.S. Pat. No. 4,551,453 include a compound having the formula

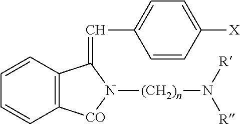

wherein X and R′ are both hydrogen atoms and R″ is a methyl or ethyl group; or R′ and R′ are both methyl or ethyl groups, or R′ and R″ together with the nitrogen atom to which they are attached may be a morpholino group, and X represents OH, OCOCH3, OCH3, OCOOC2H5, NO2 or NH2; and n may be either 2 or 3; or the hydrochloride thereof.

N-substituted 4-piperidinecarboxamide compounds such as those described in U.S. Pat. No. 5,756,520 include 1-(3-oxo-3-phenyl-propyl)-4-phenyl-piperidine-4-carbonitrile, 1-(3-hydroxy-3-phenyl-propyl)-4-phenyl-piperidine-4-carbonitrile, 1-(3-hydroxy-3-phenyl-propyl)-4-phenyl-piperidine-4-carboxylic acid, 1-(3-acetoxy-3-phenyl-propyl)-4-phenyl-piperidine-4-carboxylic acid, 1-(3-acetoxy-3-phenyl-propyl)-4-phenyl-piperidine-4-carboxamides, 1-(3-hydroxy-3-phenyl-propyl)-4-phenyl-piperidine-4-carboxamides, 1-benzyl-4-phenyl-piperidine-4-carbonitrile, 1-benzyl-4-phenyl-piperidine-4-carboxylic acid, 1-benzyl-4-phenyl-piperidine-4-carboxamides, 4-phenyl-piperidine-4-carboxamides.

N-substituted 4-phenyl-4-piperidinecarboxamide compounds, such as those described in U.S. Pat. No. 5,360,805 include ethyl 1-hexyl-4-phenyl-4-piperidine carboxylate hydrochloride, ethyl 1-[4-ethoxybutyl]-4-phenyl-4-piperidinecarboxylate hydrochloride, 1-(2-Ethoxyethyl)-4-cyano-4-phenyl-piperidine, 1-Hexyl-4-phenyl-4-piperidinecarboxic acid hydrochloride, 1-[4-Ethoxybutyl]-4-phenyl-4-piperidinecarboxylic acid hydrochloride, 1-(2-Ethoxyethyl)-4-phenyl-4-piperidine-carboxylic acid hydrochloride, N-Butyl-1-methyl-4-phenyl-4-piperidinecarboxamide and N-Ethyl-1-hexyl-4-phenyl-4-piperidinecarboxamide.

Compounds from the International patent application No. WO 95/21821 include compounds of formula

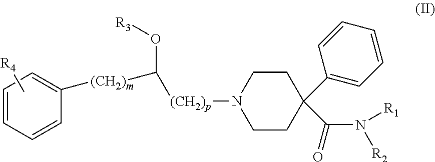

wherein a) R₁ is hydrogen or a straight or branched alkyl group with 1-3 carbon atoms, and R₂ is a straight or branched alkyl group with 1-3 carbon atoms, or b) R₁ and R₂ form together a chain —(CH₂)ₙ—, wherein n is 3, 4 or 5, or —(CH₂)₂O(CH₂)₂—; m is 0-1; p is 1-2; R₃ is hydrogen or —COCH3; and R₄ is hydrogen, —CH3, —OH or —OCH3.

In one embodiment, the local anesthetic is an amino-amide chosen among prilocaine 2-(propylamino}-2′-propionoto-luidide); etidocaine (2-ethylpropylamino-2′,6′-butyroxylidide); lidocaine (2-diethylamino-2′,6′-acetoxy-lidide), articaine, aptocaine, ropivacaine, bupivacaine (1-butyl-2′,6′-dimethyl-2-piperidinecarboxanilide), mepivacaine (I-methyl-2′,6′-pipecoloxylidide) or QX-314 (N-(2,6-Dimethylphenylcarbamoylmethyl)triethylammonium) and/or any analog and/or functional derivative thereof and/or any combination thereof.

In another embodiment the local anesthetic is an amino-amide chosen among mepivacaine or mepivacaine chlorhydrate.

In another embodiment the local anesthetic is an amino-ester chosen among ambucaine, amolanone, amylocaine, benoxinate, betoxycaine, biphenamine, butacaine, butamben, butethamine, butoxycaine, 2 chloroprocaine, cocaethylene, cocaine, cyclomethycaine, dimethocaine, piperocaine, procaine, propoxycaine, pseudococaine, risocaine, proparacaine, tetracaine, ethyl aminobenzoate, dextro-eucaine, hexylcaine, hydroxyprocaine, hydroxytetracaine, isobutylp-aminobenzoate, leucinocaine mesylate, meperidine, meprylcaine, metabutoxycaine, naepaine, orthocaine, parethoxycaine, piridocaine, propanocaine, dibucaine, benzocaine, O-aminoalkylsalicylate compounds such as those described in U.S. Pat. No. 4,298,603; and/or analog and/or functional derivative thereof and/or a mixture thereof.

Examples of O-aminoalkylsalicylate compounds are Methyl O-(3-Dimethylanilnopropyl)salicylate, Ethyl O-(2-Dimethylamino-1(and 2)-methylethyl)salicylate, Isopropyl O-(3-Dimethylaminopropyl)salicylate, n-Butyl O-(3-Dimethylaminopropyl)salicylate, n-Butyl O-(2-Dimethylaminoethyl)salicylate, n-Butyl O-(3-Piperidinopropyl)salicylate, n-Butyl O-(3-Dimethylamino-2-methylpropyl)salicylate, n-Butyl O-(2-Morpholinoethyl)salicylate, n-Butyl O-(2-Piperidinoethyl)salicylate, n-Butyl O-(2-Dimethylamino-1(and 2)-methylethyl)salicylate, n-Butyl 0-[2-(Hexamethyleneimine)-ethyl]salicylate, n-Butyl O-(2-Diethylaminoethyl)salicylate, iso-Amyl O-(3-Dimethylaminopropyl)salicylate, n-Amyl O-(3-Dimethylaminopropyl)salicylate, n-Amyl 0-[2-(Hexamethyleneimine)-ethyl]salicylate, n-Amyl O-(3-Diethylaminopropyl)salicylate, n-Amyl O-(3-Piperidinopropyl)salicylate, n-Amyl O-(2-Piperidinoethyl)salicylate, n-Amyl O-(2-Morpholinoethyl)salicylate, n-Amyl O-(2-Diisopropylaminoethyl)salicylate, n-Amyl O-(2-Dimethylamino-1(and 2)-methylethyl)salicylate, n-Amyl O-(3-Dimethylamino-2-methylpropyl)salicylate, n-Amyl O-(2-Dimethylaminoethyl)salicylate, n-Amyl O-(2-Diethylaminoethyl)salicylate, n-Hexyl O-(3-Dimethylaminopropyl)salicylate, n-Decyl O-(3-Dimethylaminopropyl)salicylate, n-Dodecyl O-(3-Dimethylaminopropyl)salicylate, n-Butyl O-(3-Dimethylaminopropyl)salicylate

In another embodiment the local anesthetic is an amino-ester chosen among cocaine, tetracaine, procaine, dibucaine or benzocaine and/or any analog and/or functional derivative thereof and/or any combination thereof.

In one embodiment, the invention relates to a composition comprising a local anesthetic chosen in the group consisting of amino amides, amino esters and any combination thereof.

In a still further embodiment, the invention relates to a pharmaceutical composition comprising a local anesthetic chosen in the group consisting of amino amides and amino esters or the above described composition and at least one pharmaceutically acceptable excipient or diluent.

In one embodiment, the composition or pharmaceutical composition of the present invention further comprises at least one additional active agent.

In one embodiment, the local anesthetic of the present invention is to be administered with at least one active agent.

In a specific embodiment, the additional active agent is a Specific Serotonin Recapture Inhibitor (SSRI).

In a specific aspect of the invention, the SSRI is chosen among citalopram, escitalopram, fluoxetine, paroxetine, sertraline and vilazodone and any combination thereof.

In a more specific embodiment, the amino amide is the mepivacaine chlorhydrate and is to be administrated from about 30 to 600 mg per each intake, more preferably from 40 to 550 mg per each intake and even more preferably from 50 to 300 mg per each intake.

According to a still further embodiment, the local anesthetic or the composition of the invention is to be administered daily, every other day, every three days, every four days, every five days, every six days, every seven days, every eight days, every nine days, every ten days, every eleven days, every twelve days, every thirteen days, every fourteen days, every fifteen days or as needed for the rest of the patient's life.

In a still further embodiment, the composition is to be administered parenterally or orally, preferably orally.

### Definitions

In the present invention, the following terms have the following meanings:
“About” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

“Amino amide”: refers to aminoamide compounds analogs and derivative thereof, their salts and their solvates, which are commonly used local anesthetics for topical, peripheral nerve, plexus, spinal and epidural anesthesia and more particularly to

bupivacaine, levo-bupivacaine, carticaine, lidocaine, prilocaine, etidocaine, levo-etidocaine, dextro-etidocaine, mepivacaine, levo-mepivacaine, ropivacaine, levo-ropivacaine, trimecaine, pyrrocaine, dibucaine, lidocaine salicylate monohydrate, octacaine, oxethazaine, pipecoloxylidides, sameridine, articaine, aptocaine, QX-314, 2-alkyl-2-alkylamino-2′, 6′acetoxylidide compounds such as those described in U.S. Pat. No. 3,862,321, tertiaryalkylamino-lower acyl-xylidide compounds such as those described in U.S. Pat. No. 3,925,469; 2(ω-alkylaminoalkyl)-3-(4-substituted-benzylidene) phthalimidine compounds or 2-(ω-dialkylaminoalkyl)-3-(4-substituted-benzylidene) phthalimidine compounds such as those described in U.S. Pat. No. 4,551,453; L-N-n-propylpipecolic acid-2,6-xylidide compounds such as those described in U.S. Pat. No. 4,695,576; N-substituted 4-piperidinecarboxamide compounds such as those described in U.S. Pat. No. 5,756,520; N-substituted 4-phenyl-4-piperidinecarboxamide compounds, such as those described in U.S. Pat. No. 5,360,805; compounds of formula (II):

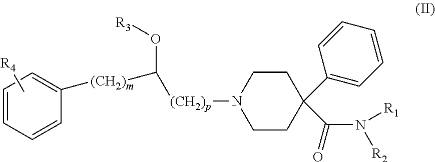

wherein R₁₋₄, m, and P are defined as in International Patent Application Publication No. WO 95/21821, and/or analog and/or functional derivative thereof and/or any combination thereof.
“Amino ester” refers to aminoesters compounds analogs and derivative thereof, their salts and their solvates which are commonly used local anesthetics for topical, peripheral nerve, plexus, spinal and epidural anesthesia and more particularly to ambucaine, amolanone, amylocaine, benoxinate, betoxycaine, biphenamine, butacaine, butamben, butethamine, butoxycaine, 2 chloroprocaine, cocaethylene, cocaine, cyclomethycaine, dimethocaine, piperocaine, procaine, propoxycaine, pseudococaine, risocaine, proparacaine, tetracaine, ethyl aminobenzoate, dextro-eucaine, hexylcaine, hydroxyprocaine, hydroxytetracaine, isobutylp-aminobenzoate, leucinocaine mesylate, meperidine, meprylcaine, metabutoxycaine, naepaine, orthocaine, parethoxycaine, piridocaine, propanocaine, dibucaine, benzocaine, O-aminoalkylsalicylate compounds such as those described in U.S. Pat. No. 4,298,603; and/or analog and/or functional derivative thereof and/or a mixture thereof.
“Analog” refers broadly to the modification or substitution of one or more chemical moieties on a parent compound and may include functional derivatives, positional isomers, tautomers, zwitterions, enantiomers, diastereomers, racemates, isosteres or stereochemical mixtures thereof.
“Brain dysfunctions” refers to consequences of stroke, tumor, head trauma, inflammatory diseases of the central nervous system, degenerative diseases, depression or psychosis but does not refer to transient dysfunctions resulting from migraine, transient ischaemic attacks, seizures, transient metabolic disorders such as hypoglycemia.
“Compound” as used throughout the specification includes but is not limited to: active metabolite, enantiomer, isomer, functional derivative, analog or salt of the amino amides and amino esters of the invention.
“Functional derivative” refers to a compound which possesses similar IC₅₀ values and kinetics properties as amino amides or amino esters. The functional derivative of the invention possesses the capacity to treat the neurological symptoms resulting from brain dysfunctions.
“Neurological symptoms” refers to motor, cognitive, sensory, visual deficits, and/or to apathetic, catatonic, vegetative, minimally conscious states, resulting from dysfunctions of the central nervous system. Within the meaning of the invention, neurological symptoms do not encompass transient neurological symptoms, which includes symptoms resulting from migraine, transient ischaemic attacks, seizures, transient metabolic disorders such as hypoglycemia.
“Pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, and general safety and purity standards as required by FDA Office of Biologics standards.
“Recovery” refers to a return to a normal state of health, mind, or strength of a patient, wherein the normal state is the state of health, mind or strength before the appearance of brain dysfunctions. Within the meaning of the invention, the recovery is “transient” when the recovery lasts the time of the pharmacological effect of the treatment. The recovery is “partial” when the patient returns to a state of health, mind or strength that is below the normal state of health, mind or strength (i.e. before the appearance of brain dysfunction).
“Subject” refers to a mammal, preferably a human. In one embodiment, the subject is female. In another embodiment, the subject is male.
“Therapeutically effective amount” means an amount of a local anesthetic, a composition or a pharmaceutical composition that provides a therapeutic benefit to an individual. In one embodiment, the term “effective amount” means level or amount of compound that is aimed at, without causing significant negative or adverse side effects to the target (1) delaying or preventing the onset of neurological symptoms resulting from brain dysfunctions; (2) slowing down or stopping the progression, aggravation, or deterioration of one or more neurological symptoms resulting from brain dysfunctions; (3) bringing about ameliorations of the neurological symptoms resulting from brain dysfunctions; (4) reducing the severity or incidence of the brain dysfunctions; or (5) curing the brain dysfunctions. A therapeutically effective amount may be administered prior to the relapse of neurological symptoms, for a prophylactic or preventive action. Alternatively, or additionally, the therapeutically effective amount may be administered after initiation of the brain dysfunctions, for a therapeutic action.
“Treatment” refers to therapeutic treatment, prophylactic or preventative measures before the relapse of neurological symptoms and deferment of the disease onset; wherein the object is to delay, prevent or slow down (lessen) the targeted pathologic condition or disorder or at least one symptom of the disorder. Those in need of treatment include those already with existing symptoms or those in whom said symptoms are to be prevented or delayed. A subject or mammal is successfully “treated” if, after receiving a dose of a composition according to the invention, the patient shows observable and/or measurable improvements, decrease in symptoms. The above parameters for assessing successful treatment and improvement in the disease are readily measurable by routine procedures familiar to a physician.

### DETAILED DESCRIPTION

This invention relates to a local anesthetic chosen in the group consisting of amino amides, amino esters and a mixture thereof for use in the treatment of neurological symptoms resulting from brain dysfunctions.

The invention also relates more particularly to a local anesthetic chosen in the group consisting of amino amides and amino esters for its use for inducing the transient and/or partial recovery of neurological symptoms resulting from brain dysfunctions.

Within the meaning of the invention, the symptoms of brain dysfunctions result from stroke, tumor, head trauma, inflammatory diseases of the central nervous system, degenerative diseases, depression or psychosis.

Examples of inflammatory diseases of the central nervous system include, without limitation, multiple sclerosis and neuromyelitis optica.

Examples of degenerative diseases include, without limitation, Alzheimer's disease, Parkinson's disease, Lewy body disease, frontotemporal lobar degeneration and motor neuron disease.

As used herein, the term “psychosis” refers to psychiatric disorders that can be associated with catatonia, such as schizophrenia and major depressive disorder.

The brain dysfunctions that are treated by the local anesthetic of the invention result in motor, cognitive, sensory, visual deficits, and/or in apathetic, catatonic, vegetative, minimally conscious states.

Motor deficit is defined as a reduction in strength. The reduction in strength will be measured by the attending physician by methods and in references to standards well known in the art. Such tests include clinical assessment with the standard clinical 0-5 rating scale (Neuroanatomy Through Clinical Cases, Blumenfeld et al., 2010) and the Fugl-Meyer scale (Fugl-Meyer, Axel R., et al. “The post-stroke hemiplegic patient. 1. a method for evaluation of physical performance.” Scandinavian journal of rehabilitation medicine 7.1 (1975): 13-31.). Examples of motor deficits include, without limitation, distal and proximal motor deficits of the upper and lower limbs, and of the muscles of the vertebral column, pharynx and mouth.

Cognitive deficit is defined as a reduction in the cognitive abilities. Tests to measure cognitive abilities are well known in the art and are part of the neurological clinical exam. Specific tests include, for example, MMSE (Mini Mental State Examination) for overall mental status, FAB (Frontal Assessment Battery) for executive functions, Five words Test for memory, AES (Apathy Evaluation Scale) for apathy, BDAE (Boston Diagnostic Aphasia Examination) for language, VOSP (Visual Object and Space Perception Battery) for vision.

Sensory deficit is defined as a reduction in the ability to perceive external stimulations applied to the body. Such reduction can be measured with standard methods of clinical assessment (Neuroanatomy Through Clinical Cases, Blumenfeld et al., 2010). Examples of sensory deficits comprise, without limitation, reduced perception of painful external stimulations, temperature, crude and light touch, and proprioception.

Visual deficits consist in a reduction in visual field and/or a reduction of the visual acuity. Visual field is assessed with methods well known in the art, which include, without limitation, the full-threshold Humphrey visual field (HVF) and 24-2 Swedish Interactive Thresholding Algorithm (SITA). Visual field is considered reduced if it shows scotoma, including quadrantanopia, hemianopia, cortical blindness. Visual acuity is assessed by the attending physician by methods and in references to standards well known in the art, which include, without limitation, use of the Snellen chart. Visual acuity is considered reduced if clinically significant, as assessed by a physician with methods and in references to standards well known in the art

As used herein, the term “minimally conscious state” refers to a condition of severely altered consciousness in which minimal but definite behavioral evidence of self or environmental awareness is demonstrated (J. T. Giacino, S. Ashwal, N. Childs, R. Cranford, B. Jennett, D. I. Katz, J. P. Kelly, J. H. Rosenberg, J. Whyte, R. D. Zafonte, N. D. Zasler, Neurology February 2002, 58 (3) 349-353).

In one embodiment, the invention relates to the treatment of motor, cognitive, sensory visual deficits, and/or in apathetic, catatonic, vegetative, minimally conscious states resulting from stroke, inflammatory diseases of the central nervous system, degenerative diseases or psychosis.

In one embodiment, the invention relates to the treatment of motor, cognitive, sensory visual deficits, and/or in apathetic, catatonic, vegetative, minimally conscious states resulting from stroke.

In one embodiment, the invention relates to the treatment of sensory deficit and/or visual deficits resulting from stroke.

In one embodiment, the invention relates to visual deficits resulting from stroke.

Amino amides and amino esters are well known and commonly used as local anesthetics for topical, peripheral nerve, plexus, spinal and epidural anesthesia.

As such, and according to the invention, the amino amide of the invention is chosen bupivacaine, levo-bupivacaine, carticaine, lidocaine, prilocaine, etidocaine, levo-etidocaine, dextro-etidocaine, mepivacaine, levo-mepivacaine, ropivacaine, levo-ropivacaine, trimecaine, pyrrocaine, dibucaine, lidocaine salicylate monohydrate, octacaine, oxethazaine, pipecoloxylidides, sameridine, articaine, aptocaine, QX-314, 2-alkyl-2-alkylamino-2′, 6′acetoxylidide compounds such as those described in U.S. Pat. No. 3,862,321, tertiaryalkylamino-lower acyl-xylidide compounds such as those described in U.S. Pat. No. 3,925,469; 2(ω-alkylaminoalkyl)-3-(4-substituted-benzylidene) phthalimidine compounds or 2-(ω-dialkylaminoalkyl)-3-(4-substituted-benzylidene) phthalimidine compounds such as those described in U.S. Pat. No. 4,551,453; L-N-n-propylpipecolic acid-2,6-xylidide compounds such as those described in U.S. Pat. No. 4,695,576; N-substituted 4-piperidinecarboxamide compounds such as those described in U.S. Pat. No. 5,756,520; N-substituted 4-phenyl-4-piperidinecarboxamide compounds, such as those described in U.S. Pat. No. 5,360,805; compounds of formula (II):

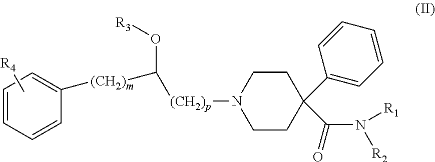

wherein R₁₋₄, m, and P are defined as in International Patent Application Publication No. WO 95/21821, and/or analog and/or functional derivative thereof, and/or any mixture thereof and the amino ester is chosen among ambucaine, amolanone, amylocaine, benoxinate, betoxycaine, biphenamine, butacaine, butamben, butethamine, butoxycaine, 2 chloroprocaine, cocaethylene, cocaine, cyclomethycaine, dimethocaine, piperocaine, procaine, propoxycaine, pseudococaine, risocaine, proparacaine, tetracaine, ethyl aminobenzoate, dextro-eucaine, hexylcaine, hydroxyprocaine, hydroxytetracaine, isobutylp-aminobenzoate, leucinocaine mesylate, meperidine, meprylcaine, metabutoxycaine, naepaine, orthocaine, parethoxycaine, piridocaine, propanocaine, dibucaine, benzocaine, O-aminoalkylsalicylate compounds such as those described in U.S. Pat. No. 4,298,603; and/or analog and/or functional derivative thereof and/or a mixture thereof.

In one embodiment, the amino amide of the invention is chosen among prilocaine, etidocaine, lidocaine, articaine, aptocaine, ropivacaine, bupivacaine, mepivacaine or QX-314 and/or analog and/or functional derivative thereof and the amino ester is chosen among cocaine, tetracaine, procaine, dibucaine, benzocaine and/or analog and/or functional derivative thereof and/or a mixture thereof.

In another specific aspect, the invention relates to a composition or a pharmaceutical composition comprising the amino amide, the amino ester of the invention or a mixture thereof and at least one pharmaceutically acceptable excipient.

In a specific aspect of the present invention the improvement of visual ability of a patient who suffered from almost complete cortical blindness following a bilateral occipital stroke is observed pursuant administration of mepivacaine, and more particularly, mepivacaine chlorhydrate.

In one embodiment, the aminoamide or the aminoester of the invention may be provided in the form of a pharmaceutically acceptable salt. Examples of such a salt include, but are not limited to, those formed with organic acids (e.g. acetic, lactic, citric, malic, formaric, tartaric, stearic, ascorbic, succinic, benzoic, methanesulfonic, toluenesulfonic, or pamoic acid), inorganic acids (e.g., hydrochloridic, nitric, diphosphoric, sulphuric, or phosphoric acid), and polymeric acids (e.g., tannic acid, carboxymethyl cellulose, polylactic, polyglycolic, or co-polymers of polylactic-glycolic acids) and/or a mixture thereof.

In another embodiment, the composition or pharmaceutical composition of the invention further comprises some excipients, such as, for example, surfactants (e.g. hydroxypropylcellulose); suitable carriers, such as, for example, solvents and dispersion media containing, for example, water, monosodium or disodium phosphate, sodium, potassium, calcium or magnesium chloride, ethanol, polyol (e.g. glycerol, propylene glycol, and liquid polyethylene glycol, and the like), suitable mixtures thereof, and vegetable oils, such as, for example, peanut oil and sesame oil; isotonic agents, such as, for example, sugars or sodium chloride; coating agents, such as, for example, lecithin; agents delaying absorption, such as, for example, aluminum monostearate and gelatin; preservatives, such as, for example, benzalkonium chloride, benzethonium chloride, chlorobutanol, thimerosal and the like; buffers, such as, for example, boric acid, sodium and potassium bicarbonate, sodium and potassium borates, sodium and potassium carbonate, sodium acetate, sodium biphosphate and the like; tonicity agents, such as, for example, dextran 40, dextran 70, dextrose, glycerin, potassium chloride, propylene glycol, sodium chloride; antioxidants and stabilizers, such as, for example, sodium bisulfite, sodium metabisulfite, sodium thiosulfite, thiourea and the like; nonionic wetting or clarifying agents, such as, for example, polysorbate 80, polysorbate 20, poloxamer 282 and tyloxapol; viscosity modifying agents, such as, for example dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose; and the like.

According to the invention, the dose of local anesthetic is administrated in a therapeutic amount. This amount is calculated in order to reach a desired result such as improvement of motor, cognitive, sensory, visual abilities, of consciousness and attention level. Methods for assessing the efficacy of the treatment are readily measurable by routing procedures familiar to a physician.

The local anesthetic, the composition or pharmaceutical composition of the invention may be administered by parenteral or oral administration. Parenteral administration may be by subcutaneous (s.c.) injection or intramuscular (i.m) injection to the subject.

In a more preferred embodiment the local anesthetic, the composition or the pharmaceutical composition of the invention is administered orally.

According to an embodiment, the local anesthetic, the composition or the pharmaceutical composition of the invention is in a form adapted for injection, preferably selected from the group comprising solutions, such as, for example, isotonic solution, saline solution, sterile aqueous solutions, dispersions, emulsions, suspensions, solid forms suitable for using to prepare solutions or suspensions upon the addition of a liquid prior to use, such as, for example, powder, freeze-dried compositions, liposomal forms and the like.

Sterile injectable solutions are prepared by incorporating the local anesthetic, the composition or the pharmaceutical composition of the invention in the required amount in the appropriate solvent with various of the other ingredients enumerated above, as required, followed by filtered sterilization. Generally, dispersions are prepared by incorporating the various sterilized composition into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

According to an embodiment, the local anesthetic, the composition or the pharmaceutical composition of the invention is in a form adapted to oral administration. According to a first embodiment, the form adapted to oral administration is a solid form selected from the group comprising tablets, pills, capsules, soft gelatin capsules, sugar-coated pills, orodispersing tablets, effervescent tablets or other solids. According to a second embodiment, the form adapted to oral administration is a liquid form, such as, for example, a drinkable solution, a buccal spray, liposomal forms and the like. The oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

It will be understood that the dose of the local anesthetic and/or additional active agent of the present invention will be decided by the attending physician within the scope of sound medical judgment. The specific dose for any particular subject will depend upon a variety of factors including the symptom being treated and the severity of the symptom; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific amino amide employed; and like factors well known in the medical arts. For example, it is well known within the skill of the art to start doses of the compound at levels lower than those required to achieve the desired therapeutic effect and to gradually increase the dosage until the desired effect is achieved.

In one embodiment of the invention, the dose of mepivacaine chlorhydrate in the composition of the invention is to be administrated from about 30 to about 600 mg per each intake, more preferably from about 40 to about 550 mg per each intake and even more preferably from about 50 to about 300 mg per each intake

In one embodiment of the invention, the dose of prilocaine chlorhydrate in the composition of the invention is to be administrated from about 0.50 mg/kg to about 0.90 mg/kg of body weight per intake.

In one embodiment of the invention, the dose of articaine chlorhydrate in the composition of the invention is of less than 7 mg/kg body weight per intake.

In one embodiment of the invention, the dose of bupivacaine chlorhydrate in the composition of the invention is of less than 150 mg/kg body weight per intake.

In one embodiment of the invention, the dose of mepivacaine chlorhydrate about 30, 31, 31, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 90, 100, 150, 200, 250 300 mg per intake.

The form of the amino amide or of the amino ester and concentration used may vary according to the patient's age and medical condition.

In one embodiment, the composition or the pharmaceutical composition of the present invention further comprises an additional active agent.

In one embodiment, the composition of the invention is to be administered alone, i.e. is not administered in combination with another therapeutic agent.

In one embodiment, the composition of the invention is to be administered with another therapeutic agent.

In a specific embodiment, this therapeutic agent is a Specific Serotonin Recapture Inhibitor (SSRI).

In a further embodiment of the invention, a synergistic effect is obtained where the amino amide or the amino ester is administered in combination with a SSRI. According to this embodiment, the time-period between two administrations can be extended.

According to this aspect the extension of the time period between two administrations can be extended by at least two, three, four, five, six, seven, eight, nine, ten, eleven, twelve, thirteen, fourteen, fifteen, sixteen, seventeen, eighteen, nineteen or twenty days.

In a specific embodiment, the SSRI is chosen among citalopram, escitalopram, fluoxetine, paroxetine, sertraline or vilazodone.

In a preferred embodiment, the SSRI chosen is paroxetine.

In one embodiment, the additional active agent, may be administered separately or in conjunction (concomitantly or sub sequentially) with the local anesthetic or the compositions of the invention.

In one embodiment, the local anesthetic, the composition or the pharmaceutical composition of the invention is administered daily, every other day, every three days, every four days, every five days, every six days, every seven days, every eight days, every nine days, every ten days, every eleven days, every twelve days, every thirteen days, every fourteen days, every fifteen days, once a month, twice a month, once a week, twice a week, at least once a day, twice, or three times a day over a period determined by the skilled man in the art such as for at least a week, at least a month, for at least two months, at least a year, or more as needed for the rest of the patient's life.

According to one embodiment of the invention, the administration dose of the local anesthetic, the composition or the pharmaceutical composition and the duration of the treatment are determined by the skilled artisan and personally adapted to each subject.

In one embodiment of the invention, the dose of the local anesthetic, the composition or the pharmaceutical composition of the invention is to be administered for a chronic treatment.

In another embodiment of the invention, the dose of the local anesthetic, the composition or the pharmaceutical composition of the invention is to be administered for an acute treatment.

### BRIEF DESCRIPTION OF THE DRAWINGS

FIG. 1 shows an assessment of the visual field with the full-threshold Humphrey visual field (HVF), 24-2 Swedish Interactive Thresholding Algorithm (SITA) (Carl Zeiss Meditec Inc., Dublin, Calif.) during on period

FIG. 2 shows an assessment of the visual field with the full-threshold Humphrey visual field (HVF), 24-2 Swedish Interactive Thresholding Algorithm (SITA) (Carl Zeiss Meditec Inc., Dublin, Calif.) during off period

FIG. 3 shows a bilateral occipital lobe infarct

FIG. 4 shows regions whose connectivity with the left calcarine cortex increased (white) or decreased (black) significantly during on—as compared to off-periods

FIG. 5 shows that the bilateral lateral occipital cortex (white), an intact part of the patient's visual cortex, increased its average functional connectivity with the rest of the brain during on—as compared to off-periods

FIG. 6 shows regions of significant increase in metabolism, as measured with 18F-FDG PET-scanner, in the intact left occipital and temporal cortex during on—as compared to off-periods.

### EXAMPLES

The present invention is further illustrated by the non-limitative following examples.

### Example 1: General Observation

In 2010, a patient underwent surgery for the removal of a cervical herniated intervertebral disc (C6-C7). Immediately following surgery, he suffered from a dissection of his right vertebral artery, resulting in bilateral occipital and right cerebellar infarcts. He presented initially with almost complete cortical blindness, left hemiparesis, gait instability, mild swallowing difficulties. Visual perception improved in a small peripheral part of his right visual hemifield, while all other symptoms recovered fully.

Apart from the patient's visual status, his general neurological examination was normal, either off or on Mepivacaine. He had no sensory or motor impairment, no abnormalities of the cranial nerves, no cognitive deficit (MMSE=29/30, FAB 16/18, Five Words Test 10/10). There was no psychiatric symptoms, except for a depressive mood whenever the patient was off medication and virtually blind.

### Example 2: Clinical Assessment of Vision

During off periods, clinical assessment of the visual field by confrontation showed complete blindness, except for a restricted region in the periphery of the right visual hemifield. During on periods, vision returned to normal in the right hemifield while dense hemianopia persisted in the left hemifield. The patient described the transition from off to on periods as a progressive widening, over a matter of seconds, of the island of spared vision in the periphery of the right visual field, to the whole right hemifield. The inverse phenomenon took place, over about 1 minute, during the transition from on to off periods.

### Example 3: Visual Field Assessment

Those clinical observations were supported and refined by assessment of the visual field with the full-threshold Humphrey visual field (HVF), 24-2 Swedish Interactive Thresholding Algorithm (SITA) (Carl Zeiss Meditec Inc., Dublin, Calif.).

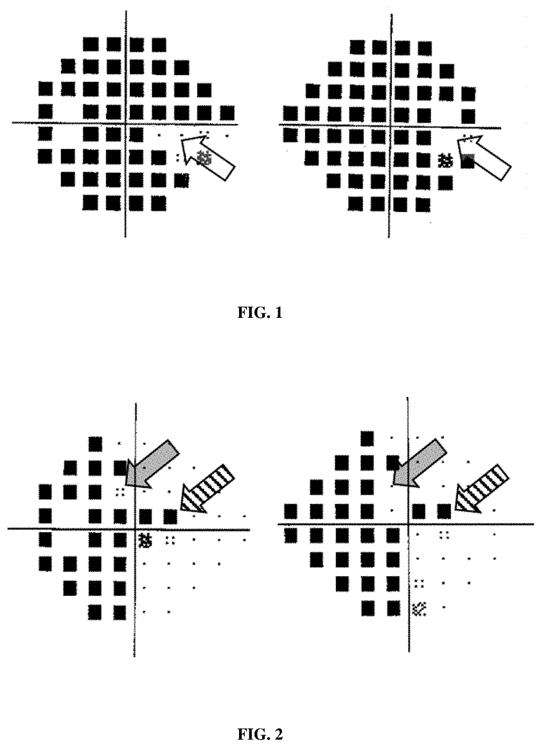

FIG. 1 shows that there was no perception even at the highest stimulation intensity (black squares) during off period (left and right eyes), except for the sparing of a right homonymous peripheral area (white arrows).

FIG. 2 shows that vision returned to normal in the whole right hemifield, except for a small superior paracentral island (striped arrows). It also recovered in a small paramedial island in the left superior quadrant (gray arrows). The same pattern prevailed using Goldmann perimetry.

### Example 4: Neurovisual Evaluation

As shown in the table below, during on periods, all tests involving visual perception were normal, except for a mild deficit in the Facial recognition test, with no discernible impairment of face perception in everyday life.

VISUOPERCEPTION, VISUOCONSTRUCTION AND
VISUOSPATIAL REASONING
Birmingham object recognition battery (BORB)
(Riddoch et Humphreys, 1993

| | |
|---|---|
| TEST 1: Copying | 9/9 |
| TEST 2: Length Match Task | 26/30 (z = 0.56) |
| TEST 3: Size Match Task | 28/30 (z = 0.29) |
| TEST 4: Orientation Match Task | 25/30 (z = 0.08) |
| TEST 5: Position of Gap Match Task | 37/40 (z = 0.48) |
| TEST 6: Overlapping Figures | — |
| TEST 7: Minimal Feature View Test | 25/25 (z = 0.85) |
| TEST 8: Foreshortened View Task | 25/25 (z = 1.31) |
| TEST 9: Drawing from Memory | 6/6 |
| TEST 10: Object Decision Task | 26/32 (z = 0.45) |
| TEST 11: Function Match Task | 31/32 (z = 0.45) |
| TEST 12: Associative Match Task | 29/30 (z = 0.63) |

Other tests

| | |
|---|---|
| Benton's Judgment of Line Orientation | 14/15 |
| Hooper Test (Richardson and Marattoli, 1996) | 27/30 (z = 1.30) |
| OD 80 picture naming test (Deloche and | 80/80 |
| Hannequin, 1996) | |
| Facial recognition test (Benton, 1963) | 38/54 |
| Block design test (WAIS-IV) | 53/66 (NS = 13) |
| Matrix reasoning test (WAIS-IV) | 22/26 (NS = 14) |

### Example 5: General Neuropsychological Status

Cognitive abilities were tested using standardized batteries. There was no impairment of attention, episodic memory, executive functioning, language, calculation.

### Example 6: Brain Imaging

1. Anatomical MRI

First, there is an infarct affecting a large part of the left mesial occipital lobe (striped arrows), including the calcarine sulcus, with cortical and white matter lesions (FIG. 3). This is the cause of the left homonymous hemianopia. Note that the ventral bank of the calcarine sulcus is less affected, with no underlying white matter FLAIR hyperintensity, which may explain why recovery under Mepivacaine extends to part of the left superior quadrant. Second, there is a smaller right occipital infarct with underlying white matter FLAIR hyperintensity (white arrow). It is located posterior and ventral relative to the calcarine sulcus, which corresponds retinotopically to the island of right visual field which is not improved by Mepivacaine.

2. Resting State Functional MRI

Resting state fMRI is a method of functional magnetic resonance imaging (fMRI) that is used in brain mapping to evaluate regional interactions that occur in a resting or task-negative state, when an explicit task is not being performed. We gathered MRI images while the patient was at rest with his eyes closed, during off and on periods. Those data were analyzed using the CONN toolbox (https://web.conn-toolbox.org/home). We report here two results providing pathophysiological correlates of vision recovery in the patient's right visual hemifield under Mepivacaine.

a. Changes in Connectivity of the Left Primary Visual Cortex

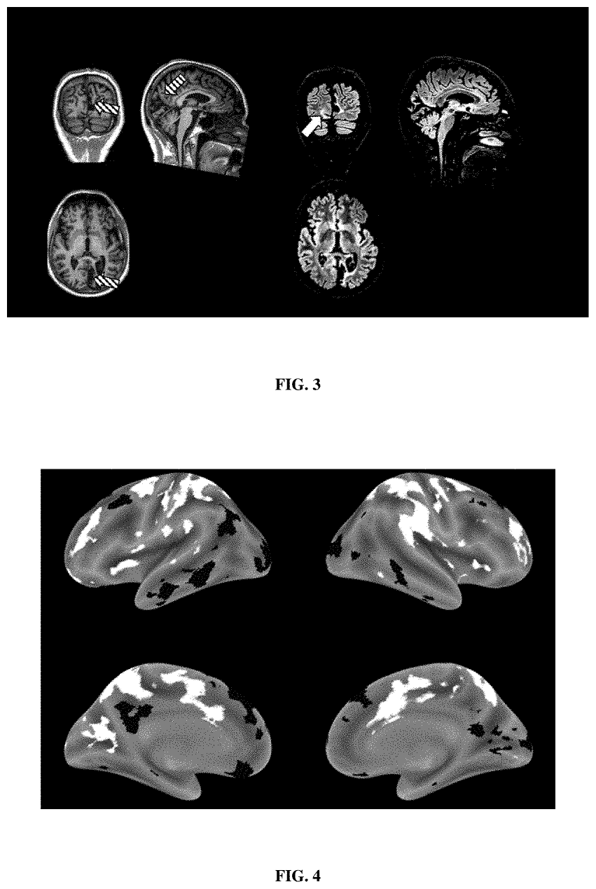

The left early visual cortex (or calcarine cortex) is the entry gate into the brain of visual information from the right hemifield. We reasoned that this area, which showed little structural damage on anatomical MRI, should recover its function under Mepivacaine. We predicted that whatever its underlying mechanism, functional recovery implies a restoration of functional communication between the left calcarine cortex and distant cortical areas. As shown in FIG. 4, the left calcarine cortex showed an increase in connectivity with bilateral frontoparietal regions, corresponding to a network controlling attention to external events (Corbetta and Shulman, 2002). Conversely, there was a decrease in correlation with a set of mesial and lateral regions, corresponding to the so-called default-mode network involved in internally oriented cognitive processes (Raichle et al., 2001). As a control, we performed the same analysis from the symmetrical right-hemispheric calcarine region, which showed no significant differences between on and off periods.

b. Changes in Intrinsic Connectivity

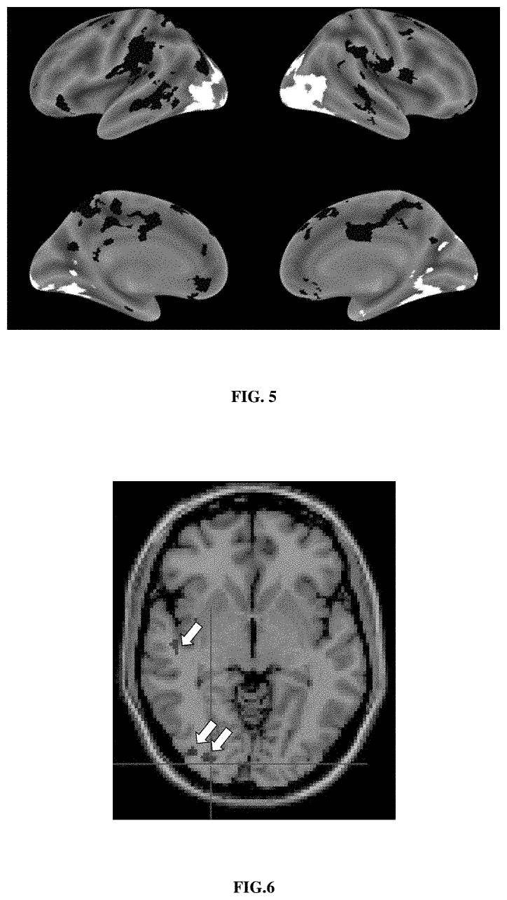

We predicted that the restoration of vision should be accompanied with an overall increase of connectivity between the visual system and the rest of the brain. We computed for each voxel of the brain the average connectivity with the rest of the brain (or “intrinsic connectivity”), and studied the changes of this value between off and on periods. As shown in FIG. 5, the bilateral lateral occipital cortex (white), an intact part of the patient's visual cortex, indeed increased its functional connectivity with the rest of the brain during on—as compared to off-periods

3. 18F-FDG PET-Scan

We observed (1) a severe and constant hypometabolism in the severely lesioned right mesial occipital cortex; and (2) regions of significant increase in metabolism in the intact left occipital and temporal cortex, during on—as compared to off-periods (FIG. 6).

1-16. (canceled)

17. A method of treatment of neurological symptoms resulting from brain dysfunctions comprising administering a local anesthetic chosen from the group consisting of amino amides, amino esters and a combination thereof.

18. The method of treatment of claim 17, comprising inducing the transient and/or partial recovery of neurological symptoms resulting from brain dysfunctions.

19. The method of treatment of claim 17, wherein the symptoms of brain dysfunctions result from stroke, tumor, head trauma, inflammatory diseases of the central nervous system, degenerative diseases, depression or psychosis.

20. The method of treatment of claim 17, wherein the brain dysfunctions result in motor, cognitive, sensory, visual deficits, and/or in apathetic, catatonic, vegetative, minimally conscious states.

21. The method of treatment of claim 17, wherein the amino-amide is chosen among bupivacaine, levo-bupivacaine, carticaine, lidocaine, prilocaine, etidocaine, levo-etidocaine, dextro-etidocaine, mepivacaine, levo-mepivacaine, ropivacaine, levo-ropivacaine, trimecaine, pyrrocaine, dibucaine, lidocaine salicylate monohydrate, octacaine, oxethazaine, pipecoloxylidides, sameridine, articaine, aptocaine, QX-314, 2-alkyl-2-alkylamino-2′, 6′acetoxylidide compounds, tertiaryalkylamino-lower acyl-xylidide; 2(ω-alkylaminoalkyl)-3-(4-substituted-benzylidene) phthalimidine compounds or 2-(ω-dialkylaminoalkyl)-3-(4-substituted-benzylidene) phthalimidine compounds; L-N-n-propylpipecolic acid-2,6-xylidide compounds; N-substituted 4-piperidinecarboxamide compounds; N-substituted 4-phenyl-4-piperidinecarboxamide compounds, compounds of formula (II):
wherein a) R1 is hydrogen or a straight or branched alkyl group with 1-3 carbon atoms, and R2 is a straight or branched alkyl group with 1-3 carbon atoms, or b) R1 and R2 form together a chain —(CH2)n-, wherein n is 3, 4 or 5, or —(CH2)2O(CH2)2-; m is 0-1; p is 1-2; R3 is hydrogen or —COCH3; and R4 is hydrogen, —CH3, —OH or —OCH3,
and/or analog and/or functional derivative thereof and/or a mixture thereof.

22. The method of treatment of claim 21, wherein the amino-amide is mepivacaine or mepivacaine chlorhydrate.

23. The method of treatment of claim 17, wherein the amino ester is chosen among ambucaine, amolanone, amylocaine, benoxinate, betoxycaine, biphenamine, butacaine, butamben, butethamine, butoxycaine, 2 chloroprocaine, cocaethylene, cocaine, cyclomethycaine, dimethocaine, piperocaine, procaine, propoxycaine, pseudococaine, risocaine, proparacaine, tetracaine, ethyl aminobenzoate, dextro-eucaine, hexylcaine, hydroxyprocaine, hydroxytetracaine, isobutylp-aminobenzoate, leucinocaine mesylate, meperidine, meprylcaine, metabutoxycaine, naepaine, orthocaine, parethoxycaine, piridocaine, propanocaine, dibucaine, benzocaine, O-aminoalkylsalicylate compounds; and/or analog and/or functional derivative thereof and/or a mixture thereof.

24. The method of treatment of claim 17, wherein the local anesthetic chosen from the group consisting of amino amides and amino esters and a mixture thereof.

25. The method of treatment of claim 17, further comprising administering another active agent.

26. The method of treatment of claim 25, wherein said active agent is at least one Specific Serotonin Recapture Inhibitor (SSRI); preferably the SSRI is paroxetine.

27. The method of treatment of claim 26, wherein the SSRI is chosen among citalopram, escitalopram, fluoxetine, paroxetine, sertraline or vilazodone or any combination thereof.

28. The method of treatment of claim 17, wherein the amino amide is the mepivacaine chlorhydrate and is to be administrated from about 30 to about 600 mg per each intake, more preferably from about 40 to about 550 mg per each intake and even more preferably from about 50 to about 300 mg per each intake.

29. The method of treatment of claim 17, wherein the local anesthetic is to be administered daily, every other day, every three days, every four days, every five days, every six days, every seven days, every eight days, every nine days, every ten days, every eleven days, every twelve days, every thirteen days, every fourteen days, every fifteen days, once a month, twice a month, once a week, twice a week, at least once a day, twice, or three times a day over a period determined by the skilled man in the art such as for at least a week, at least a month, for at least two months, at least a year, or more on as needed basis for the rest of the patient's life.

30. The method of treatment of claim 17, wherein the local anesthetic is to be administered parenterally or orally, more preferably orally.

31. The method of treatment of claim 26, wherein the local anesthetic is administrated to a subject in need thereof concomitantly or in conjunction: separately or sub sequentially with a SSRI.
